(19) United States
(12) Patent Application Publication
(10) Pub. No.: US 2008/0051381 A1
(43) Pub. Date: Feb. 28, 2008

(54) Suppression of estrus in mares by a single injection method

(76) Inventor: Patrick J. Burns, Lexington, KY (US)

Correspondence Address: ROETZEL & ANDRESS, 1375 EAST 9TH STREET, CLEVELAND, OH, 44114, US

(21) Appl. No.: 11/507,772

(22) Filed: Aug. 22, 2006

(51) Int. Cl. A61K 31/56; A61K 31/57

(52) U.S. Cl. 514/177

(57) ABSTRACT

The present invention generally relates to methods of suppressing estrus in breeding or competitive animals comprising parenterally administering to breeding animals an estrus suppressing amount of 17-α-Allyl-17-β-hydroxyoestra-4,9,11-trien-3-one. According to the method of the present invention, estrus can be suppressed for any of a variety of periods including 30 days or more. Furthermore, the method of the present invention includes repeated sustained release doses for an indefinite period.

### FIELD OF THE INVENTION

The present invention is in the general field of veterinary methods. More particularly, the present invention generally relates to methods for suppressing estrus.

### BACKGROUND OF THE INVENTION

Progestins are frequently used to prevent the expression of estrus in race, show, and broodmares for periods of a week to a month or longer. Unfortunately, daily treatments can be an impractical method to administer Altrenogest or progesterone to mares. Particularly, daily administrations are inconvenient, time consuming, and costly to owners and are stressful to the animals. Recent advances in biodegradable controlled release drug delivery systems offer the potential for single administration products to replace prolonged daily treatment protocols. Such formulations would reduce labor and the associated handling stress to the animals and producers, and offer veterinarians an important means of maintaining effective compliance rates on farms and show barns with wide varieties of management systems.

Although Altrenogest and progesterone have been unequivocally demonstrated to suppress estrus and ovulation in mares, other widely prescribed synthetic progestins including medroxyprogesterone acetate have been shown not to effect estrus, ovulation or pregnancy maintenance.

Thus, there is a substantial gap in the art. Particularly, the art lacks a method for suppressing estrus with a single, sustained release injection. Moreover, there is a need in the art for a method of suppressing estrus in mares that entails less time and or cost commitments. The present invention addresses this and other needs in the art.

### SUMMARY OF THE INVENTION

The present invention generally relates to methods for suppressing estrus in any of a variety of animals including equine. Methods within the scope of the present invention include those which are effective in suppressing estrus with a single dose or with limited doses administered over widely spaced intervals. In some embodiments, animals to which the present method may be applied include equine, porcine, canine, bovine, ovine, and caprine.

The present invention also relates to a method of suppressing estrus in breeding, competitive or show mammals comprising a single controlled release parenterally administered effective amount of synthetic progestin 17-α-Allyl-17-β-hydroxyoestra-4,9,11-trien-3-one.

### BRIEF DESCRIPTION OF THE FIGURES

FIG. 1 is a plot showing mean injection site scores for each treatment on days 0, 1, 3, 6, and 9.

### DETAILED DESCRIPTION

The present invention generally relates to methods for suppressing estrus in any of a variety of animals including equine. Methods within the scope of the present invention include those which are effective in suppressing estrus with a single dose or with limited doses administered over widely spaced intervals. In some embodiments, animals to which the present method may be applied include equine, porcine, canine, bovine, ovine, and caprine.

Altrenogest is also known as 17-α-Allyl-17-β-hydroxyoestra-4,9,11-trien-3-one, allyltrenbolone, and Regumate. Altrenogest has been assigned CAS No. 850-52-2. As used herein, the term Altrenogest includes the compound noted above as well as any suitable formulation that includes the compound. Altrenogest can be administered in any of a variety of sustained-release preparations, such as liquid formulations, microparticle formulations, or any combination thereof. Average daily sustained-release dosages of Altrenogest within the scope of the present invention include 1 to 100 mg/day, 5 to 50 mg/day, or even 10 to 40 mg/day. Here, as elsewhere in the specification and claims, ranges may be combined. Dosages may be formulated to release Altrenogest over any of a variety of periods including between 1 and 30 days, 5 to 20 days or even 10 to 15 days. In some embodiments, sustained release may continue for more than one month. In addition to single sustained release dosages, the method of the present invention can also include multiple sustained release dosages. For example, a formulation that is effective at suppressing estrus for thirty days, can be administered every thirty days. Furthermore, there is no limit to the number of times that the formulation can be administered.

The following example demonstrates the efficacy of the present invention. Thirty one randomly selected light-horse mares are maintained on native pasture and ad libitum hay. Verification of normal reproductive cyclicity is determined by follicular ultrasound, teasing with a vigorous stallion, and radioimmunoassay of plasma progesterone concentrations. Mares displaying estrus (i.e. the estrus mare group) with plasma progesterone levels below 1 ng/mL are followed by ultrasound until a follicle 30 mm or greater is determined. When a follicle reaches 30 mm, 2 mL of 2000 IU human chorionic gonadotropin is administered to induce ovulation. Mares in the estrus group are subsequently followed by ultrasound until ovulation and corpus luteal formation are verified. Six days after ovulation is determined, mares in this group are given a 2 mL injection of dinoprost tromethamine and randomly assigned to a treatment regime set forth in Table 1.

Mares not displaying estrus (i.e. the non-estrus mare group) with progesterone concentrations above 1 ng/mL and the presence of a corpus luteum verified by ultrasound are given 2 mL dinoprost tromethamine. Once estrus is detected by teasing with a stallion, these mares are treated as the mares assigned to the estrus mare group. Day of treatment (d0) is simultaneous with the final, mid-diestrus injection of dinoprost tromethamine (five or six days after ovulation). Mares are then randomly allotted to one of five treatments set forth in Table 1.

TABLE 1
Sustained Release Progestin Treatments

| Treatments | Total | | Dose |
|---|---|---|---|
| (treatments follow 2 mL | Dose | Dose/day | Volume |
| dinoprost tromethamine) | (mg) | (mg/d) | (mL) |
| 1) Controls (2 cc dinoprost tromethamine | — | — | — |
| only) | | | |
| 2) Medroxyprogesterone acetate as a 5 mL | 1000 | 33.3 | 5 |
| solution. Administered for 30 days | | | |
| 3) Altrenogest LA150 as a 1.5 mL solution. | 225 | 22.5 | 1.5 |
| Administered for 10 days | | | |
| 4) Altrenogest LA150 as a 3 mL solution | 450 | 30 | 3 |
| Administered for 15 days | | | |
| 5) Altrenogest MP 500 as lactide-glycolide | 500 | 16.6 | 7 |
| microparticles suspended in | | | |
| 7 mL). Administered for 30 days | | | |

The mares of each of the groups in Table 1 are followed via ultrasound every other day after treatment until a follicle 25 mm or greater is detected. Once a follicle exceeds 25 mm, the mare is scanned daily until the follicle ovulates or regressed to less than 25 mm. Blood samples are collected via jugular venipuncture before each ultrasound examination for later hormone assays. Once a mare returns to estrus and ovulates, or ovulates a large dominant follicle without showing estrus she is discontinued.

Injection site assessments are made on days 0, 1, 3, 6, and 9 post-treatment. Scores are based on a subjective scale of 0-3. Zero means no swelling; one means slight diameter swelling (i.e. about 12.5 mm); 2 means moderate diameter swelling (i.e. about 12.5 to 25 mm); and 3 means significant swelling (i.e. more than 25 mm). Plasma concentrations of progesterone are measured with commercially available reagents (Diagnostic Systems Laboratories, Webster, Tex.). Intra- and interassay CV and assay sensitivities are 5%, 8%, and 0.1 ng/mL. Data are analyzed by the Proc Mixed procedure of SAS (SAS Institute Inc., Cary, N.C.). Single point variables are analyzed via a one way ANOVA. Data from injection site scores are analyzed for effects of treatment, time, and treatment by time interactions with repeated measures. When a significant F is detected (P<0.05), the least significant difference (LSD) test is used to determine differences between groups within times.

Each of the single injection Altrenogest formulations are effective at extending (P<0.05) the return to estrus and interovulatory interval in mares when compared to controls, as shown in Table 2. The Altrenogest MP 500 formulation suppresses estrus and inhibits ovulation the longest (P<0.05). The mean days of return to estrus is 32.8±4.8 days compared to 3.9±0.5 days in the control mares. Days to ovulation relative to administration of dinoprost tromethamine and Altrenogest for the MP 500 formulation is 34.7±3.7 days compared to 8.3±1.2 days in control mares. Both doses of the liquid Altrenogest LA 150 formulation effectively extends the return to estrus following treatment (12.7±0.4 days and 15.8±1.5 days respectively) and results in a mean days to ovulation relative to treatment of 17.2±0.75 and 21.8±0.54 days for the 1.5 mL and 3 mL doses, respectively. Medroxyprogesterone acetate treatment is not effective at delaying estrus or ovulation.

TABLE 2
Days to Estrus and Ovulation following Treatment

| Formulation | N | Days to estrus | Days to ovulation |
|---|---|---|---|
| LA 150 1.5 cc | 6 | 12.7 ± 0.42^{b} | 17.2 ± 0.75^{b} |
| LA 150 3 cc | 6 | 15.8 ± 1.51^{b} | 21.8 ± 0.54^{b} |
| MP 500 | 6 | 32.8 ± 4.80^{c} | 34.7 ± 3.72^{c} |
| Medroxyprogesterone | 6 | 6.2 ± 1.38^{a} | 11.3 ± 2.14^{a} |
| Controls | 7 | 3.9 ± 0.51^{a} | 8.3 ± 1.17^{a} |

^{c}Means ± SE; means with different superscripts differ (P < 0.05)

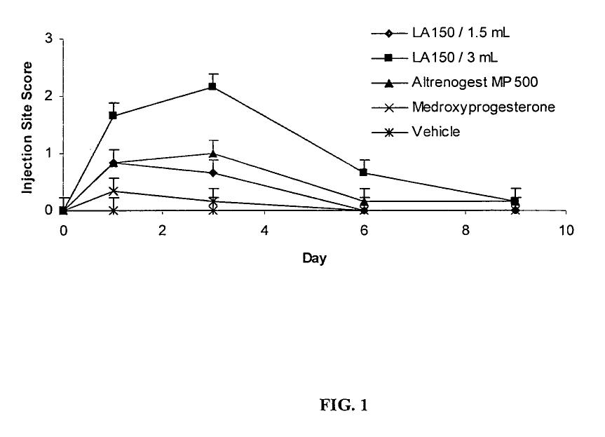

Injection site swelling is observed with all 5 formulations when compared to controls (FIG. 1) but tends to vary by mare and formulation. The Altrenogest MP500 and Altrenogest LA150/1.5 mL formulations have similar injection site swelling scores, namely only a slight swelling (i.e. score of 1). Medroxyprogesterone is the most biocompatible formulation in the mare.

Each of the embodiments set forth herein are effective at extending the interovulatory interval and delaying estrus to varying degrees. The Altrenogest MP 500 formulation has the greatest inhibitory effect with only slight swelling. This formulation can be beneficial for performance horses by inhibiting estrus for a period of 30 days and can be administered repeatedly as desired to maintain reproductive quiescence or anestrus. The Altrenogest LA150/1.5 mL embodiment is also very effective and can be valuable when shorter periods of estrus suppression (12 to 14 days) are desired. For example, this embodiment can be valuable in transitional mares to establish normal cycles or for estrus and ovulation synchronization programs. In some cases, this embodiment can be particularly valuable when the degree of estrus and ovulation compare favorably with daily Altrenogest treatment or progesterone plus estradiol treatments.

The embodiments described herein are examples of compositions, structures, systems and methods having elements corresponding to the elements of the invention recited in the claims. This written description enables one of ordinary skill in the art to make and use embodiments having alternative elements that likewise correspond to the elements of the invention recited in the claims. The scope thus includes compositions, structures, systems and methods that do not differ from the literal language of the claims, and further includes other compositions, structures, systems and methods with insubstantial differences from the literal language of the claims. While only certain features and embodiments have been illustrated and described herein, many modifications and changes may occur to one of ordinary skill in the relevant art. The appended claims are intended to cover all such modifications and changes.

I claim:

1. A method of suppressing estrus in breeding, competitive or show mammals comprising the step of parenterally administering a single controlled release effective amount of synthetic progestin 17-α-Allyl-17-β-hydroxyoestra-4,9,11-trien-3-one.

2. The method of claim 1 wherein the breeding, competitive or show mammals are pre or post pubescent fillies or mares.

3. The method of claim 1 wherein the breeding mammals are pubescent mares.

4. The method of claim 1 wherein the administration comprises a single sustained-release intramuscular injection providing an average daily dose of about 1 to 100 mg/day.

5. The method of claim 1 wherein the administration comprises a single sustained-release intramuscular injection providing an average daily dose of about 5 to 50 mg/day.

6. The method of claim 1 wherein the administration comprises a single sustained-release intramuscular injection, wherein the injection is repeated at about 10 to 40 day intervals.

7. The method of claim 1 wherein the administration comprises a single sustained-release intramuscular injection, wherein the injection is repeated at about 10 to 30 day intervals.

8. The method of claim 7 wherein the interval is about 30 days.

9. The method of claim 8 wherein the breeding, competitive, or show mammal is a pubescent mare.

10. The method of claim 1, wherein the administration is repeated at least once.

11. The method of claim 10, wherein the administration is repeated at or near the end of the estrus-suppressing period of the preceding dose.

12. The method of claim 1, wherein the mammal is selected from equine, porcine, bovine, canine, ovine, and caprine.

13. The method of claim 1, wherein the synthetic progestin 17-α-Allyl-17-β-hydroxyoestra-4,9,11-trien-3-one comprises a component of a liquid formulation.

14. The method of claim 1, wherein the synthetic progestin 17-α-Allyl-17-β-hydroxyoestra-4,9,11-trien-3-one comprises a component of a microparticle formulation.

15. A method of suppressing estrus in mares comprising the step of parenterally administering a single controlled release liquid formulation with an effective amount of synthetic progestin 17-α-Allyl-17-β-hydroxyoestra-4,9,11-trien-3-one, capable of releasing about 1 to 100 mg/day of the progestin over a period of about 10 to 30 days.

16. The method of claim 15, wherein the parenteral administration is an intramuscular injection.

17. The method of claim 15, wherein the progestin is capable of releasing from about 10 to 35 mg/day over a period of about 25 to 30 days.

18. The method of claim 15, wherein the progestin is capable of releasing from about 10 to 30 mg/day over a period of about 10 to 15 days.

19. The method of claim 15, wherein the administration is repeated at least once at or near the estrus-suppressing period of the preceeding dose.
